(19) United States
(12) Patent Application Publication
(10) Pub. No.: US 2025/0138248 A1
(43) Pub. Date: May 1, 2025

(54) MULTI-OPTICAL AND ELECTRICAL CONNECTOR

(71) Applicant: ABIOMED, Inc., Danvers, MA (US)

(72) Inventors: Alexander Ship, Danvers, MA (US); Dustin Seth West, Danvers, MA (US); Qingchao Kong, Danvers, MA (US); Mazen Yatim, Danvers, MA (US)

(73) Assignee: ABIOMED, Inc., Danvers, MA (US)

(21) Appl. No.: 18/927,177

(22) Filed: Oct. 25, 2024

Related U.S. Application Data
(60) Provisional application No. 63/545,703, filed on Oct. 25, 2023.
(60) Provisional application No. 63/603,514, filed on Nov. 28, 2023.

(51) Int. Cl. G02B 6/38

(52) U.S. Cl. CPC G02B 6/3817; G02B 6/3821; G02B 6/3849

(57) ABSTRACT

A plug for a blood pump may be provided. The plug may include a housing. A central optical ferrule, coaxial with a central axis of the housing, may extend partially through the housing. A first end of the central optical ferrule may be an axial distance from an outer surface at a first end of the housing. The plug may include a plurality of electrical pins, each pin disposed parallel to the central axis and independently at a radial distance from the central axis. Each electrical pin may have an end extending through the housing and extending beyond the first end of the central optical ferrule. The plug may include additional optical ferrule(s) extending at least partially though the housing. Each additional ferrule being parallel to the central axis and disposed at a radial distance from the central axis.

### CROSS-REFERENCE TO RELATED APPLICATIONS

The present application claims priority to U.S. Provisional Patent Applications Nos. 63/545,703 Filed Oct. 25, 2023, and 63/603,514 Filed Nov. 28, 2023, each of which is incorporated by reference herein by its entirety.

### TECHNICAL FIELD

The present application is drawn to optical and electrical connectors, and specifically to multifunctional hybrid connectors for mechanical circulatory support devices.

### BACKGROUND

Mechanical circulatory support devices are growing more complex over time, regularly adding new functionality. Such additional functionality requires new connections (electrical, optical, etc.), and new controllers to ensure such devices perform the additional functions correctly. However, such new controllers also preferably need to remain backwards compatible with existing support devices.

### BRIEF SUMMARY

In various aspects, a plug for a blood pump may be provided. The plug may include a first housing. The plug may include a central optical ferrule. The central optical ferrule may be coaxial with a central axis of the first housing. The central optical ferrule may extend partially through the first housing. A first end of the central optical ferrule may be an axial distance A1 from an outer surface at a first end of the first housing. A1 may be greater than 0.

The plug may include a plurality of electrical pins. Each pin may be disposed parallel to the central axis and may be disposed, independently, at a radial distance R1 from the central axis. R1 may be greater than 0. Each electrical pin may have an end extending through the first housing and extending beyond the first end of the central optical ferrule. In some embodiments, the plurality of electrical pins may include, e.g., 10-15 electrical pins.

The plug may include at least one additional optical ferrule. The additional optical ferrule(s) may extend at least partially though the housing. Each additional ferrule may have a first end an axial distance A2 from the outer surface at the first end of the first housing. A2 may be greater than 0. Each additional ferrule may be parallel to the central axis and disposed at a radial distance R2 from the central axis. R2 may be greater than 0. In some embodiments, R2 may be less than R1.

In some embodiments, the first housing may be configured to be operably coupled to a second housing at a second end of the first housing, the second being opposite the first end of the first housing. A radially-outer surface of the second housing may include at least one groove or channel configured to receive a rotatable cover when the plug is fully inserted into a receptor.

A plane defining an outer surface of the first housing through which each electrical pin extends may be parallel to and offset axially from a plane defining the first end of the central optical ferrule.

In some embodiments, the at least one additional optical ferrule may be a single additional optical ferrule. The at least one additional optical ferrule may be a plurality of additional optical ferrules.

In some embodiments, the plug may further include an additional spring. The additional spring may be operably coupled to each additional optical ferrule. In some embodiments, a spring rate of each additional spring may be configured to have a spring rate such that 3-4 mm of displacement results in a compression force of 0-20 N. In some embodiments, the plug may further include a feature. The feature may be configured to cause the at least one additional optical ferrule to retract. The feature may be configured to cause the at least one additional optical ferrule to extend. The plug may further include at least one recess in the first housing. The at least one recess may be configured to receive an optical ferrule of a receptor.

In various aspects, a receptor for a plug for a blood pump may be provided.

The receptor may include a first housing defining a central axis from a first end to a second end. The first housing may have a circular inner surface at the first end configured to receive a plug. The first housing may have an inner surface at the first end configured to receive a plug, where the inner surface may have at least one portion that may be a non-circular inner surface. The non-circular inner surface may include at least one flat surface. The non-circular inner surface may define a geometric shape. The non-circular inner surface may define a non-geometric shape.

The receptor may include a second housing coupled to the second end. The receptor may include a central optical receiver. The central optical receiver may include a central optical ferrule having a first end and a second end. The central optical ferrule may be coaxial with a central axis of the first housing and may extend through the first housing. The receptor may include a central spring disposed between a second end of the central optical ferrule and the second housing. The second housing may be configured to screw onto the second end of the first housing. A force necessary to compress the central spring may be different from a force necessary to compress at least one additional spring.

The second housing may be a split housing containing a slit. The second housing may be configured to slidably receive an optical fiber through the slit. The second housing may include a snap-fit for a bend relief of one or more optical fibers.

The receptor may include a plurality of electrical pin receivers extending at least partially through the first housing. Each electrical pin receiver may be disposed parallel to the central axis. Each electrical pin receiver may be disposed, independently, at a radial distance d1 from the central axis. d1 may be greater than 0. The plurality of electrical pin receivers may include 10-15 electrical pin receivers.

The receptor may include at least one additional optical receiver. Each additional optical receiver may include an additional optical ferrule extending though the first housing. Each additional ferrule may be parallel to the central axis and disposed at a radial distance d2 from the central axis. d2 may be greater than 0. d2 may be less than d1. The at least one additional optical receiver may be a single additional optical receiver. The at least one additional optical receiver may be a plurality of additional optical receivers. The second housing may be configured to partially cover an opening in the first housing in which the at least one additional optical receiver may be disposed. The central optical ferrule may be a different size or shape than at least one of any additional optical ferrule(s). At least one ferrule of the central optical ferrule and/or additional optical ferrule(s) may be a prepolished ferrule. The receptor may include an index matching gel operably coupled to the at least one ferrule.

The receptor may include an additional spring disposed between a second end of the additional optical ferrule and at least a portion of the second housing. The receptor may include a central spacer. The central spacer may be disposed between the central spring and the second housing. Each additional optical receiver may include an additional spacer between the additional spring and the second housing.

The receptor may include a third housing coupled to the second end of the first housing. The third housing may be disposed around the second housing. The third housing may be disposed around the second housing and the second end of each electrical pin receptor.

A radially-outer surface may include a threaded portion. An adjustable nut may be operably coupled to the threaded portion.

The receptor may include a shutter disposed in an optical path of an optical fiber extending into the central optical ferrule. The optical fiber may include a hybrid optical fiber.

The receptor may include a potting material around an optical fiber, where an end of the optical fiber may be polished. One optical fiber may extend through each optical ferrule. A plurality of optical fibers may extend through the central optical ferrule and/or at least one additional optical ferrule.

In some embodiments, a spring rate of each additional spring may be configured to have a spring rate such that 3-4 mm of displacement results in a compression force of 0-20 N. In some embodiments, the receptor may further include a feature. The feature may be configured to cause the at least one additional optical ferrule to retract. The feature may be configured to cause the at least one additional optical ferrule to extend. In some embodiments, the receptor may further include at least one recess in the first housing. The at least one recess may be configured to receive an optical ferrule of a plug.

In various aspects, a system may be provided. The system may include a plug as disclosed herein removably coupled to a receptor as disclosed herein. Each electrical pin of the plug may be operably coupled to one of the plurality of electrical pin receivers of the receptor. An optical fiber disposed within the central optical ferrule of the plug may be operably coupled to an optical fiber disposed within the central optical ferrule of the receptor. An optical fiber disposed within one of the additional optical ferrules of the plug may be operably coupled to an optical fiber disposed within one of the additional optical ferrules of the receptor. The plug may be operably coupled to a blood pump. The receptor may be operably coupled to a controller configured to control the blood pump.

In some embodiments, the system may further include a dongle. The dongle may be disposed between the plug and the receptor.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a system incorporating a cardiac support system.

FIG. 2A is a side-view cut-away of a plug.

FIG. 2B is a front view of a plug.

FIG. 2C is an illustration of an exploded view of a plug.

FIG. 3A is a side-view cut-away of a receptor.

FIG. 3B is a front view of a receptor.

FIG. 3C is an illustration of an exploded view of a receptor.

FIG. 4 is an illustration of a plug coupled to a receptor.

FIGS. 5A and 5B are illustrations of a passive retraction mechanism with a single-fiber plug (5A) and multi-fiber plug (5B).

FIGS. 6 and 7 are illustrations of active retraction mechanisms.

FIG. 8 is an illustration showing a dongle between a plug and receptor.

FIG. 9 is an illustration showing a receptor that includes a recess.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

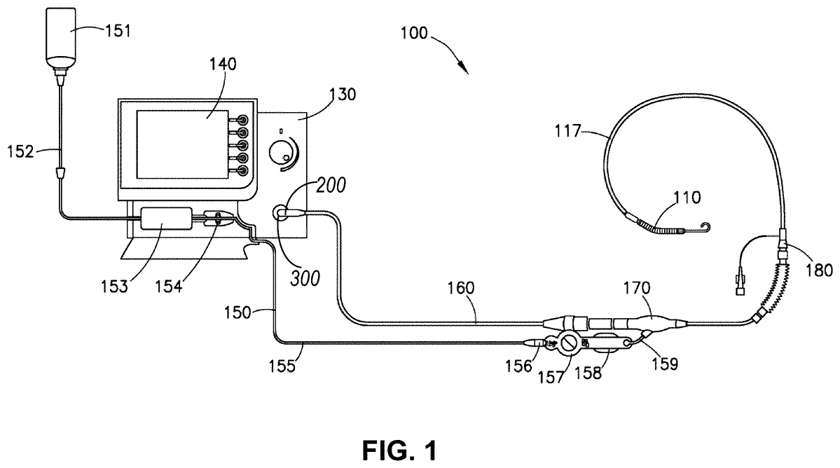

A circulatory support device (also referred to herein as a “blood pump” or simply a “pump”) may include a percutaneous, catheter-based device that provides hemodynamic support to the heart of a patient. As shown in FIG. 1, blood pump 110 may form part of a cardiac support system 100. Cardiac support system 100 also may include a controller 130 (e.g., an Automated Impella Controller*, referred to herein as an “AIC,” from ABIOMED, Inc, Danvers, Mass), a display 140. a purge subsystem 150, a connector cable 160, a plug 170, and a repositioning unit 180. As shown, controller 130 may include display 140. Controller 130 may be configured to monitor and control operation of blood pump 110. During operation, purge subsystem 150 may be configured to deliver a purge fluid to blood pump 110 through catheter tube 117 to prevent blood from entering the motor (not shown) of the blood pump. In some implementations, the purge fluid may include a dextrose solution (e.g., 5% dextrose in water with 25 or 50 IU/mL of heparin, although the purge solution need not include heparin in all embodiments). In other embodiments, the purge fluid may include a sodium bicarbonate solution. Connector cable 160 may provide an electrical connection between blood pump 110 and controller 130. Plug 170 may connect catheter tube 117, purge subsystem 150, and connector cable 160. In some implementations, plug 170 may include a storage device (e.g., a memory) configured to store, for example, operating parameters to facilitate transfer of the patient to another controller if needed. Repositioning unit 180 may be used to reposition blood pump 110 in the patient's heart.

As shown in FIG. 1, a cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to blood pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of blood pump 110 and purge disc 154. Controller 130 may also be configured to control operation of the motor (not shown) of the blood pump 110 and purge cassette 153. As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the blood pump 110 (e.g., one or more pressure sensors) and controller 130. Motor cables (not shown) within catheter tube 117, connector cable 160, and plug 170 may provide an electrical connection between the motor of the blood pump 110 and controller 130. During operation, controller 130 may be configured to receive measurements from one or more pressure sensors of the blood pump 110 through the sensor cables (e.g., optical fibers) and to control the electrical power delivered to the motor of the blood pump 110 through the motor cables. By controlling the power delivered to the motor of the blood pump 110, controller 130 may be operable to control the speed of the motor.

Various modifications can be made to cardiac support system 100 and one or more of its components. For instance, one or more additional sensors may be added to blood pump 100. In another example, a signal generator may be added to blood pump 100 to generate a signal indicative of the rotational speed of the motor of the blood pump 110. As another example, one or more components of cardiac support system 100 may be separated. For instance, display 140 may be incorporated into another device in communication with controller 130 (e.g., wirelessly or through one or more electrical cables).

A blood pump (e.g., blood pump 110) may include a pressure sensor (e.g., an optical pressure sensor) configured to detect a pressure within the aorta of a patient's heart when the blood pump is properly positioned. The pressure signal sensed by the pressure sensor may be used, at least in part, to determine correct positioning of the blood pump within the patient's heart and/or to determine a blood flow rate through the blood pump when in operation. For instance, the pressure signal may be used in combination with a motor current signal received from a motor current sensor (not shown) and a set of stored values to determine a flow rate of blood through the blood pump. The differential pressure across the aortic valve may also indirectly be determined based on the pressure signal measuring the pressure in the aorta and the set of stored values.

The cardiac support system 100 may be coupled to the controller 130 via a plug 200 (shown here at a proximal end of connector cable 160). The controller 130 has a receptor 300 configured to receive the plug 200. These plugs and receptors may be configured to allow various signals and information to be passed between the controller and various elements of the cardiac support system.

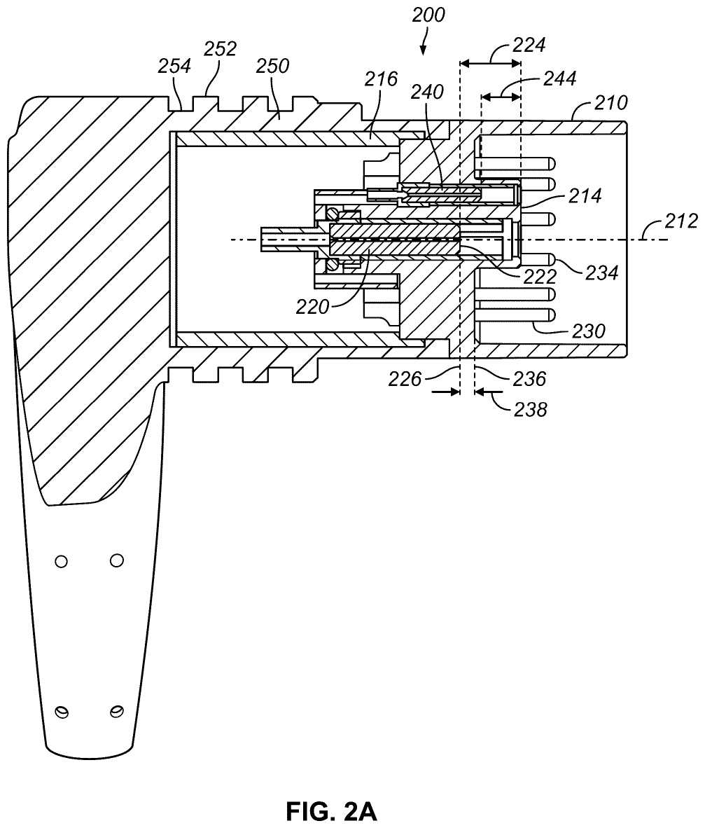

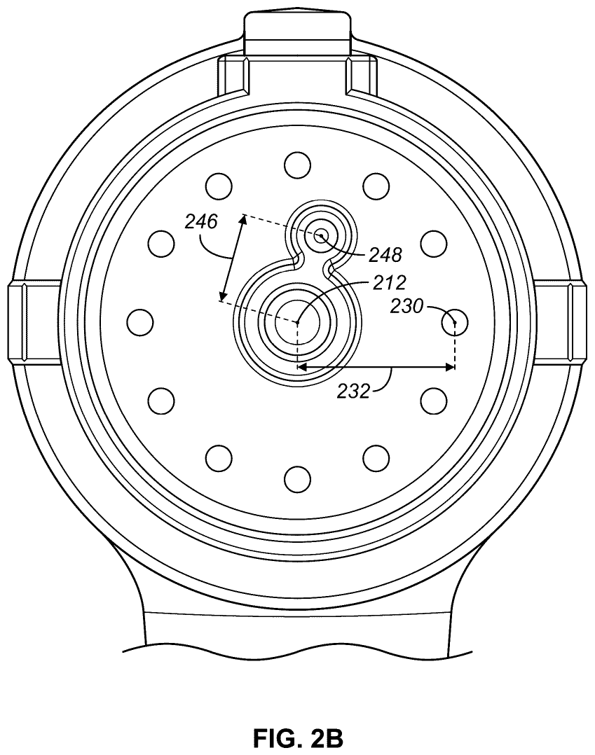

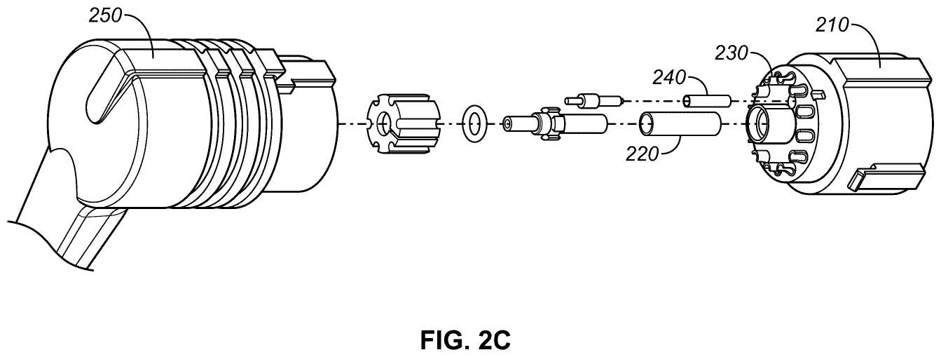

In various aspects, a plug for a blood pump may be provided. Referring to FIGS. 2A-2C, the plug 200 may include a first housing 210. The plug may include a central optical ferrule 220. The central optical ferrule may be coaxial with a central axis 212 of the first housing. The central optical ferrule may extend partially through the first housing. A first end 222 of the central optical ferrule may be an axial distance 224 (“A1”) from an outer surface at a first end of the first housing. The axial distance A1 may be greater than 0 in some embodiments.

The plug may include a plurality of electrical pins 230 (see, e.g., FIG. 2A). Each pin may be disposed parallel to the central axis and may be disposed, independently, at a radial distance 232 (“R1”) from the central axis. The radial distance R1 may be greater than 0 in some embodiments. Each electrical pin may have an end 234 extending through the first housing and extending beyond the first end 222 of the central optical ferrule. The plurality of electrical pins may include, e.g., 10-15 electrical pins. A plane 236 defining an outer surface of the first housing through which each electrical pin extends may be parallel to and offset axially (e.g., offset a distance 238 that is greater than 0) from a plane 226 defining the first end of the central optical ferrule. The electrical pins may be configured to be arranged radially symmetrically around the central optical ferrule (e.g., FIG. 2B). The electrical pins may be configured to be arranged radially asymmetrically around the central optical ferrule.

The plug may include at least one additional optical ferrule 240. The additional optical ferrule(s) may extend at least partially though the housing. Each additional ferrule may have a first end an axial distance 244 (“A2”) from the outer surface at the first end 214 of the first housing 210. In some embodiments, the axial distance A2 may be greater than 0. Each additional ferrule may be parallel to the central axis and may be disposed a radial distance 246 (“R2”) from the central axis (e.g., a central axis of the additional optical ferrule 248 may be disposed a radial distance 246 from the central axis 212 of the first housing). In some embodiments, the radial distance R2 may be greater than 0. Radial distance R2 may be less than radial distance R1 in some embodiments. The at least one additional optical ferrule may be a single additional optical ferrule (e.g., FIG. 2B). The at least one additional optical ferrule may be a plurality of additional optical ferrules. In one preferred embodiment, the plug may include three total optical ferrules (e.g., a first and second additional optical ferrule).

The first housing 210 may be configured to be operably coupled to a second housing 250 (see FIG. 2A) at a second end 216 of the first housing, the second end 216 being opposite the first end 214 of the first housing. A radially-outer surface 252 of the second housing may include at least one groove or channel 254 configured to receive a rotatable cover (not shown) when the plug is fully inserted into a receptor. In some embodiments, the rotatable cover may be configured to prevent the plug from inadvertently coming out of the receptor. In such embodiments, the rotatable cover may be configured to be rotated out of the groove or channel when the plug is being removed from the receptor.

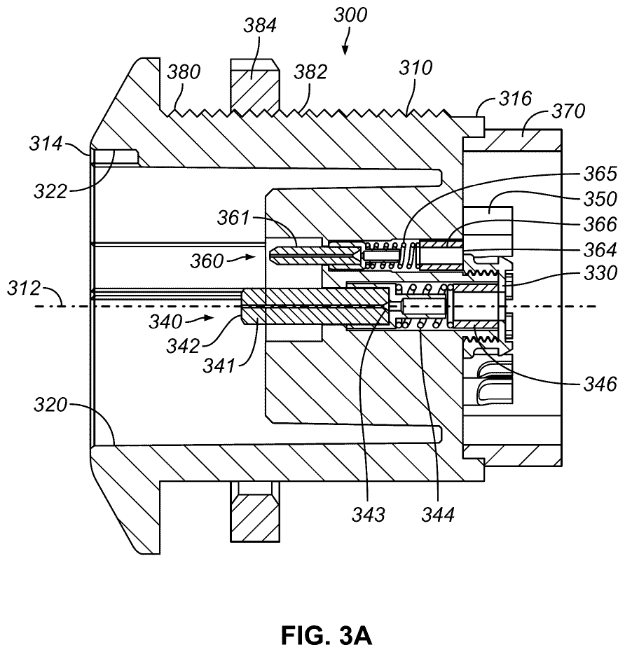

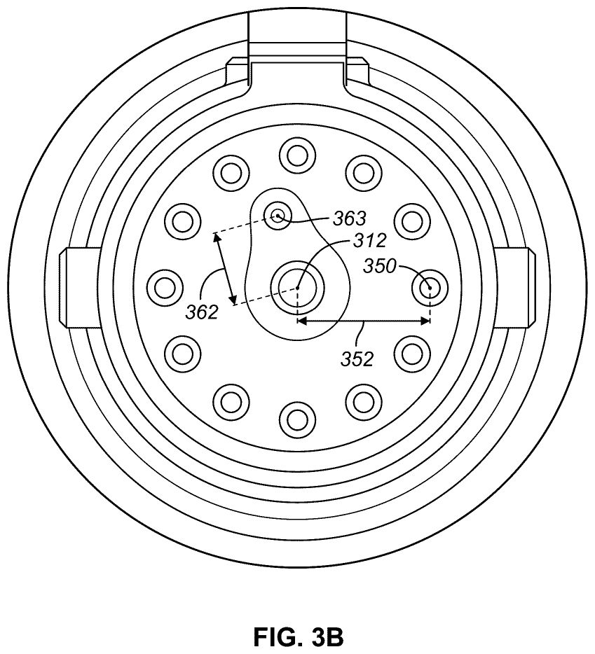

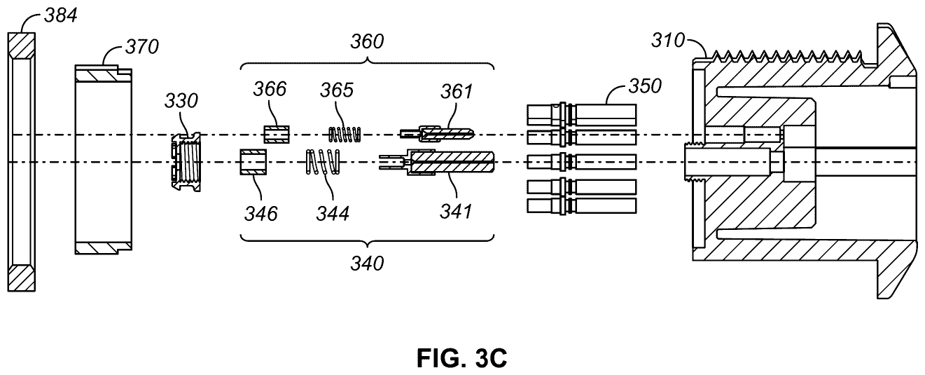

In various aspects, a receptor for a plug for a blood pump may be provided. Referring to FIGS. 3A-3C, a receptor 300 may include a first housing 310 defining a central axis 312 from a first end 314 to a second end 316. The first housing may have an inner surface 320 at the first end configured to receive a plug. The inner surface may be circular. The first housing may have an inner surface at the first end configured to receive a plug, where the inner surface may have at least one portion 322 that is a non-circular inner surface. The non-circular inner surface may include at least one flat surface. The non-circular inner surface may define a geometric shape. The non-circular inner surface may define a non-geometric shape.

The receptor may include a second housing 330 coupled to the second end 316. The receptor may include a central optical receiver 340. Referring to FIG. 3C, the central optical receiver 340 may include a central spacer 346, a central spring 344, and a central optical ferrule 341. The central optical receiver may include a central optical ferrule 341 having a first end 342 and a second end 343. The central optical ferrule may be coaxial with a central axis 312 of the first housing and may extend through the first housing. The receptor may include a central spring 344 disposed between a second end of the central optical ferrule and the second housing. The second housing may be configured to screw onto the second end of the first housing. A force necessary to compress the central spring may be different from a force necessary to compress at least one additional spring.

The second housing may be a split housing containing an axial slit. The second housing may be configured to slidably receive an optical fiber through the slit. The second housing may include a snap-fit for a bend relief of one or more optical fibers.

The receptor may include a plurality of electrical pin receivers 350 extending at least partially through the first housing. Each electrical pin receiver may be disposed parallel to the central axis. Each electrical pin receiver may be disposed, independently, at a radial distance 352 (“d1”) from the central axis 312. In some embodiments, the radial distance d1 may be greater than 0. The plurality of electrical pin receivers may include 10-15 electrical pin receivers. The electrical pin receivers may be configured to be arranged radially symmetrically around the central optical ferrule. In some embodiments, the arrangement of electrical pin receivers may be configured to match an arrangement of electrical pins of a plug as disclosed herein.

The receptor may include at least one additional optical receiver 360. Each additional optical receiver may include an additional optical ferrule 361 extending though the housing. Each additional ferrule may be parallel to the central axis and disposed at a radial distance 362 (“d2”) from the central axis 312 (e.g., a central axis 363 of additional optical ferule 361 may be a radial distance 362 from the central axis 312 of the first housing) (see FIG. 3B). In some embodiments, the radial distance d2 may be greater than 0. The radial distance d2 also may be less than radial distance d1. The at least one additional optical receiver may be a single additional optical receiver. The at least one additional optical receiver may be a plurality of additional optical receivers. The second housing 330 may be configured to partially cover an opening 364 in the first housing 310 in which the at least one additional optical receiver 360 is disposed.

The central optical ferrule may be a different size or shape than at least one of any additional optical ferrule(s). At least one ferrule of the central optical ferrule and/or additional optical ferrule(s) may be a prepolished ferrule. The receptor may include an index matching gel operably coupled to the at least one ferrule.

Referring to FIG. 3C, the at least one additional optical receiver 360 may include an additional spring 365 disposed between a second end of the additional optical ferrule and at least a portion of the second housing. The at least one additional optical receiver may include a central spacer 346. The central spacer may be disposed between the central spring 344 and the second housing 330. Each additional optical receiver may include an additional spacer 366 between the additional spring and the second housing.

The receptor may include a third housing 370 coupled to the second end of the first housing. The third housing may be disposed around the second housing. The third housing may be disposed around the second housing and the second end of each electrical pin receptor.

A radially-outer surface 380 may include a threaded portion 382. An adjustable nut 384 may be operably coupled to the threaded portion.

In some embodiments, the receptor may include a shutter (not shown) disposed in an optical path of an optical fiber extending into the central optical ferrule. In some embodiments, the shutter may be on the controller, and may be adjusted (e.g., rotated) to cover the opening when the plug is removed. The optical fiber may include a hybrid optical fiber. The receptor may include a potting material around an optical fiber, where an end of the optical fiber is polished. One optical fiber may extend through each optical ferrule. A plurality of optical fibers may extend through the central optical ferrule and/or at least one additional optical ferrule.

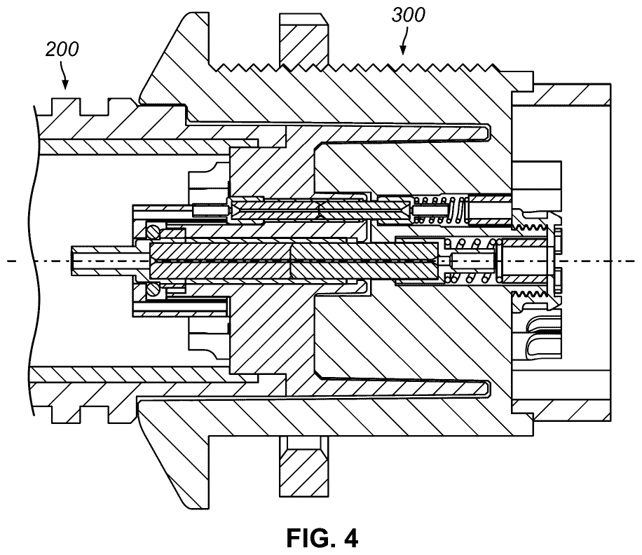

In various aspects, a system may be provided. Referring to FIG. 4, the system may include a plug 200 as disclosed herein removably coupled to a receptor 300 as disclosed herein. Each electrical pin of the plug may be operably coupled to one of the plurality of electrical pin receivers of the receptor. An optical fiber disposed within the central optical ferrule of the plug may be operably coupled to an optical fiber disposed within the central optical ferrule of the receptor. An optical fiber disposed within one of the additional optical ferrules of the plug may be operably coupled to an optical fiber disposed within one of the additional optical ferrules of the receptor. The plug may be operably coupled to a blood pump. The receptor may be operably coupled to a controller configured to control the blood pump.

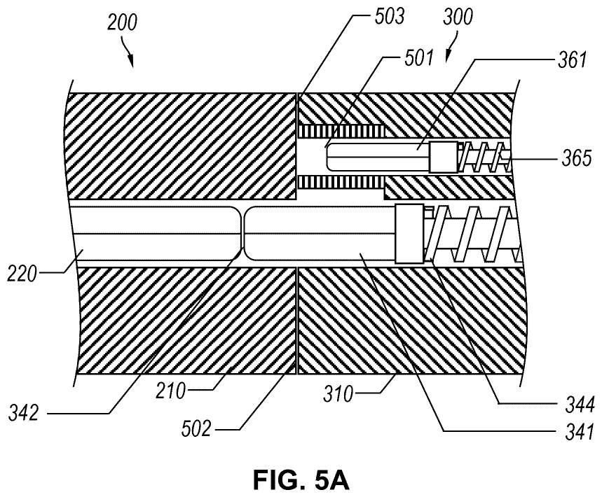

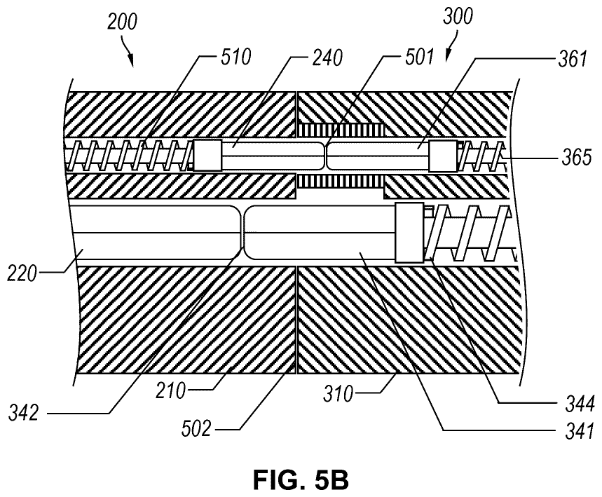

In various aspects, the plug/receptor combination may utilize passive retraction to allow a multi-fiber component (e.g., the plug or receptor) to connect with either a single- or multi-fiber component (e.g., the other of the plug or receptor). As will be appreciated, such passive retraction mechanisms may be integrated into the plug, the receptor, or both. Referring to FIG. 5A, a single fiber plug 200 is shown, with a first housing 210 and a central optical fiber ferrule 220. A multi-fiber receptor 300 is shown with a first housing 310, a central optical fiber ferrule 341 and an additional optical ferrule 361.

As shown, there may be a central spring 344 and an additional spring 365 to control the positioning and force exerted on or by the central optical fiber ferrule and the additional optical ferrule, respectively. As shown, the central optical fiber ferrule of the receptor has a first end 342 that may be operably coupled with the central optical ferrule of the plug. The first end 342 is shown as being disposed within the first housing of the plug (e.g., extending beyond a contacting face 502 of the first housing 310 of the receptor that is in contact with the first housing 210 of the plug). As will be understood, the positioning of the first end 342 of the central optical ferrule may be easily adjusted, e.g., such that the end is disposed within the first housing of the receptor.

The additional optical ferrule 361 may have an end 501 opposite the end contacting the additional spring 365 such that the end 501 is disposed within the first housing of the receptor. As shown, the end 501 is not in contact with a contacting surface 503 of the plug (e.g., a surface of the plug that is in contact with the first housing of the receptor). In some embodiments, the additional spring may be configured to have a lower spring rate than the central spring. In some embodiments, the additional spring may be configured to have a spring rate such that 3-4 mm of displacement results in a compression force of about 0-20 N. As will be understood, the springs, etc., are shown as being in the receptor, but could easily be incorporated into the plug in other embodiments.

FIG. 5A shows a single fiber plug coupled to a multi-fiber receptor. When a multi-fiber plug is coupled to that same multi-fiber receptor, the additional optical fiber ferrules can interact. As shown in FIG. 5B, an additional spring 510 in the plug 200 may be operably coupled to an additional optical ferrule 240 in the plug. The additional springs in the plug and/or the receptor may be configured to achieve a target compression force for the chosen fiber optic termination. For example, the compression force may be between 0 N and 20 N, although other suitable compression forces may be used. In some embodiments, the additional spring in the plug has a spring rate equivalent to the spring rate of the additional spring in the receptor. In some embodiments, the additional spring in the plug has a spring rate greater than the spring rate of the additional spring in the receptor. In some embodiments, the additional spring in the plug may have a spring rate less than the spring rate of the additional spring in the receptor. In various aspects, the plug/receptor combination may utilize active retraction to allow a multi-fiber component (e.g., the plug or receptor) to connect with either a single- or multi-fiber component (e.g., the other of the plug or receptor). In this manner, the plug may be couplable to a receptor even if there are different numbers of fibers components in the plug and receptor. That is, the plug and/or receptor may include one or more features that (automatically or via user intervention) mechanically slide the additional optical features out of the way to facilitate connection to single-fiber receptors.

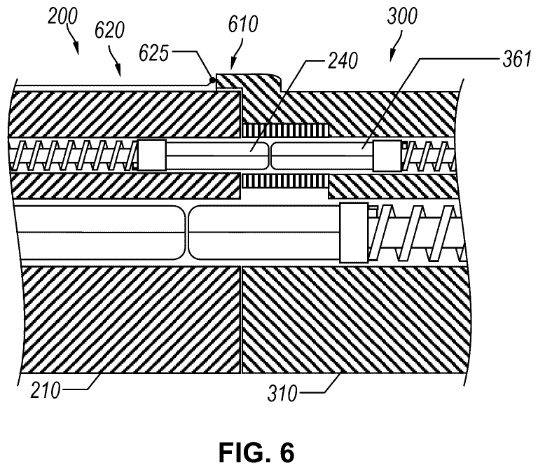

Referring to FIG. 6, an embodiment is shown wherein a receptor feature 610, such as a tab or protrusion, is configured to interact with a plug feature 620, such as an axially sliding component with a tab 625 or other element configured to contact receptor feature 610. In FIG. 6, when a multi-fiber receptor receives a multi-fiber plug, the receptor feature 610 may interact with the plug feature 620 automatically. Here, the plug feature may slide axially backwards (e.g., away from the receptor), which directly or indirectly allows the additional optical ferrule 240 of the plug to slide forward and contact the additional optical ferrule 361 of the receptor. As will be appreciated, other types of active retraction mechanisms may be used in other embodiments.

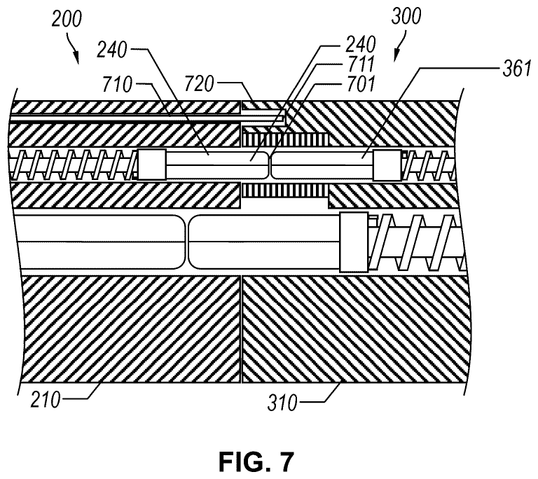

Referring to FIG. 7, an embodiment is shown wherein a plug feature 710 is configured to extend into a receptor feature 720 (here, a cavity or void in first housing 310 of the receptor) when a multi-fiber receptor is utilized. In some embodiments, an end 711 of the plug feature 710 extends further than the end 701 of the additional optical ferrule 240 of the plug. In this way, if no cavity or void exists in the first housing, indicating the receptor is a single-fiber receptor, the plug feature 710 will slide backwards, which may directly or indirectly cause the additional optical ferrule 240 to slide backwards (e.g., away from the receptor), preventing the additional optical ferrule from being damaged or interfering with the plug/receptor connection.

As will be recognized, the plug features could also be slid backwards manually by a user. Further, it will be understood that the features could be reversed, so the features described as plug features are on the receptor instead, and vice-versa.

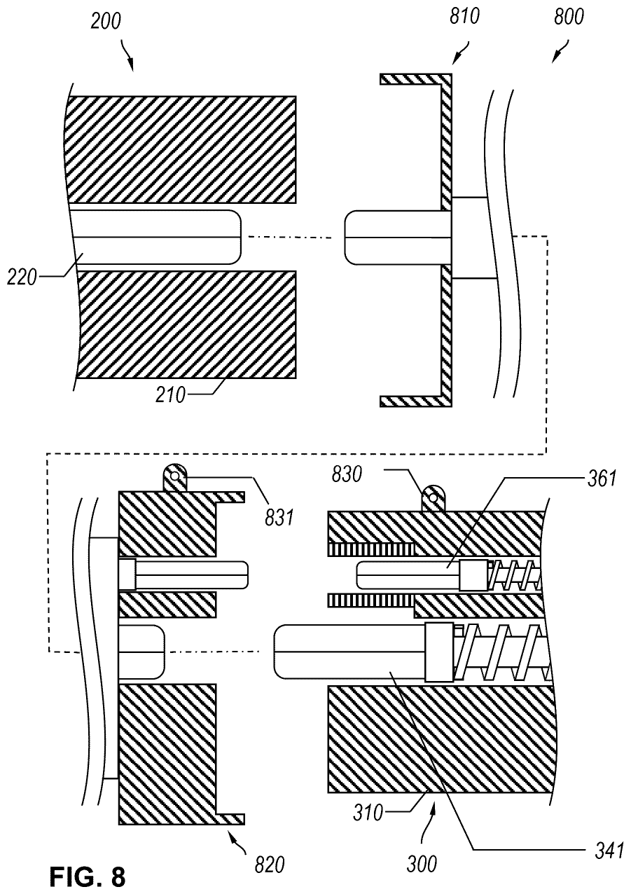

Rather than a plug and receptor directly connecting, a dongle (or adapter) may be utilized. As seen in FIG. 8, a dongle 800 may be configured to have a first end 810 and a second end 820. The first end may be configured to interface with the plug 200 while the second end may be configured to interface with the receptor 300. In some embodiments, such as the one shown in FIG. 8, one end (here, first end 810) may be configured to interface with a single-fiber plug or receptor, while the other end (here, second end 820) may be configured to interface with a multi-fiber plug or receptor. This may allow the plug to be used with connectors even when there are different numbers of fibers in the plug on each. In some embodiments, both ends only include a single ferrule. In some embodiments, a feature 830 on the receptor or plug is configured allow the dongle to be permanently attached (e.g., via a chain, cord, etc.) to the receptor or plug. In some embodiments, the dongle may include a feature 831 configured to allow the chain, cord, etc., to be attached to the dongle.

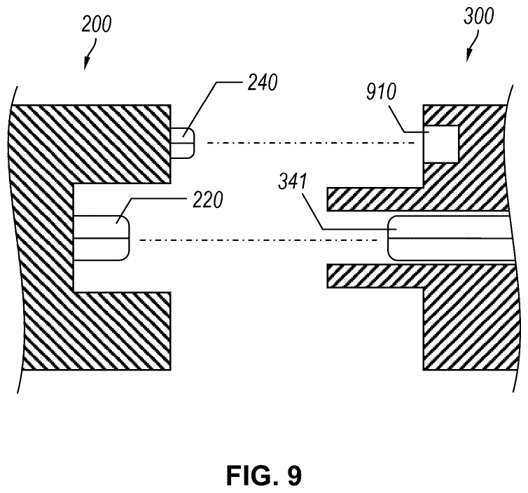

In various aspects, a plug or receptor may include a recess that permits connection to receptors or plugs with different or more optical fibers. Referring to FIG. 9, receptor 300 with a single optical ferrule (e.g., central optical ferrule 341) may have one or more recesses 910. While the central optical ferrule 341 may be configured to be operably coupled to a central optical ferrule 220 of plug 200, there may be no additional optical ferrule in the receptor to operably coupled with the additional optical ferrule 240 of the plug. Here, instead, the additional optical ferrule 240 may be configured to extend into the recess 910 of the receptor. In some embodiments, the recess may be filled with air. In some embodiments, the recess may include a soft and clean material. In some embodiments, the soft and clean material may be compressible. In some embodiments, the soft and clean material may be configured as rods that can bend or flex.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A plug for a blood pump, comprising:
a first housing;
a central optical ferrule, coaxial with a central axis of the first housing, extending partially through the first housing, a first end of the central optical ferrule being an axial distance A1 from an outer surface at a first end of the first housing, where A1>0;
a plurality of electrical pins, each pin disposed parallel to the central axis and independently at a radial distance R1 from the central axis, where R1>0, each electrical pin having an end extending through the first housing and extending beyond the first end of the central optical ferrule; and
at least one additional optical ferrule extending at least partially though the housing, each additional ferrule having a first end an axial distance A2 from the outer surface at a first end of the first housing, where A2>0, each additional ferrule being parallel to the central axis and disposed at a radial distance R2 from the central axis, where R2>0.

2. The plug of claim 1, wherein the first housing is configured to be operably coupled to a second housing at a second end of the first housing, the second end being opposite the first end of the first housing.

3. The plug of claim 2, wherein a radially-outer surface of the second housing includes at least one groove or channel configured to receive a rotatable cover when the plug is fully inserted into a receptor.

4. The plug of claim 1, wherein the plurality of electrical pins includes 10-15 electrical pins.

5. The plug of claim 1, wherein the at least one additional optical ferrule is a single additional optical ferrule.

6. The plug of claim 1, wherein the at least one additional optical ferrule is a plurality of additional optical ferrules.

7. The plug of claim 1, wherein each R2<R1.

8. The plug of claim 1, wherein a plane defining an outer surface of the first housing through which each electrical pin extends is parallel to and offset axially from a plane defining the first end of the central optical ferrule.

9. The plug of claim 1, further comprising an additional spring operably coupled to each additional optical ferrule.

10. The plug of claim 9, wherein a spring rate of each additional spring is configured to have a spring rate such that 3-4 mm of displacement results in a compression force of 0-20 N.

11. The plug of claim 1, further comprising a feature configured to cause the at least one additional optical ferrule to retract.

12. The plug of claim 1, further comprising a feature configured to cause the at least one additional optical ferrule to extend.

13. The plug of claim 1, further comprising at least one recess in the first housing configured to receive an optical ferrule of a receptor.

14. A receptor for a plug for a blood pump, comprising:
a first housing defining a central axis from a first end to a second end;
a second housing coupled to the second end;
a central optical receiver including:
a central optical ferrule having a first end and a second end, the central optical ferrule being coaxial with a central axis of the first housing and extending through the first housing; and
a central spring disposed between a second end of the central optical ferrule and the second housing;
a plurality of electrical pin receivers extending at least partially through the first housing, each electrical pin receiver disposed parallel to the central axis and independently at a radial distance d1 from the central axis, where d1>0; and
at least one additional optical receiver, each additional optical receiver including:
an additional optical ferrule extending though the first housing, each additional ferrule being parallel to the central axis and disposed at a radial distance d2 from the central axis, where d2>0; and
an additional spring disposed between a second end of the additional optical ferrule and at least a portion of the second housing.

15. The receptor of claim 14, further comprising a central spacer disposed between the central spring and the second housing.

16. The receptor of claim 14, wherein each additional optical receiver further comprises an additional spacer between the additional spring and the second housing.

17. The receptor of claim 14, further comprising a third housing coupled to the second end of the first housing, the third housing disposed around the second housing.

18. The receptor of claim 17, wherein the third housing is disposed around the second housing and the second end of each electrical pin receptor.

19-40. (canceled)

41. The receptor of claim 14, wherein one optical fiber extends through each optical ferrule.

42. The receptor of claim 14, wherein a plurality of optical fibers extend through the central optical ferrule and/or at least one additional optical ferrule.

43-46. (canceled)

47. A system, comprising:
a plug of claim 1 removably coupled to a receptor of claim 14;
wherein each electrical pin of the plug is operably coupled to one of the plurality of electrical pin receivers of the receptor;
wherein an optical fiber disposed within the central optical ferrule of the plug is operably coupled to an optical fiber disposed within the central optical ferrule of the receptor; and
wherein an optical fiber disposed within one of the additional optical ferrules of the plug is operably coupled to an optical fiber disposed within one of the additional optical ferrules of the receptor.

48-49. (canceled)
